(19) United States
(12) Patent Application Publication
(10) Pub. No.: US 2007/0184443 A1
(43) Pub. Date: Aug. 9, 2007

(54) Streptococcus pneumoniae knockout mutants

(75) Inventor: Antonello Covacci, Siena (IT)

Correspondence Address: NOVARTIS VACCINES AND DIAGNOSTICS INC., CORPORATE INTELLECTUAL PROPERTY R338, P.O. BOX 8097, Emeryville, CA, 94662-8097, US

(73) Assignee: CHIRON SRL, Siena (IT)

(21) Appl. No.: 10/567,570

(22) PCT Filed: Aug. 9, 2004
(86) PCT No.: PCT/IB04/02709

§ 371 Date: Oct. 18, 2006

(30) Foreign Application Priority Data
Aug. 8, 2003 (GB) 0318688.9

(51) Int. Cl. C12Q 1/68; G01N 33/554; C12N 1/21

(52) U.S. Cl. 435/6; 435/7.32; 435/252.3

(57) ABSTRACT

91 genes have been identified in Streptococcus pneumoniae that, when knocked out, result in a lethal phenotype. A further 10 genes have been identified that, when knocked out, result in poor growth characteristics when cultured in the absence of blood. These 101 genes are essential to bacterial growth and are thus useful antibiotic targets. Their invention includes knockout mutants for these 101 genes and screening methods involving the protein products of the 101 genes.

### TECHNICAL FIELD

This invention relates to mutants of the bacterium Streptococcus pneumoniae (‘pneumococcus’), and to the use of pneumococcal proteins in screening methods.

### BACKGROUND ART

Streptococcus pneumoniae is a Gram-positive spherical bacterium. It is the most common cause of acute bacterial meningitis in adults and in children over 5 years of age.

It is an object of the invention to provide materials for improving the prevention, detection and treatment of S. pneumoniae infections. More specifically, it is an object of the invention to provide mutants of S. pneumoniae in which specific genes have been inactivated, and to provide specific genes and gene products from S. pneumoniae for use as targets for anti-pneumococcal drugs.

### DISCLOSURE OF TIER INVENTION

Genome sequences of several strains of S. pneumoniae are available, including those of 23F [1], 670 [2], R6 [3,4] and TIGR4 [5, 6]. Functional annotations of inferred coding sequences within these genome sequences are also available. Knowledge of sequence and/or annotation, however, does not necessarily reveal the importance of a gene product in the life cycle of pneumococcus, or the suitability of the gene product as a target for pharmaceutical intervention.

In the S. pneumoniae TIGR4 strain, 91 genes (see Table 1) have been identified which, when knocked out, result in a lethal phenotype. A further 10 genes (Table 2) have been identified which, when knocked out, result in poor growth characteristics when cultured in the absence of blood. These 101 genes are essential to bacterial growth and are thus useful antibiotic targets.

Nomenclature

As mentioned above, genome sequences of several strains of S. pneumoniae are available. Genes are referred to below by a name “SPnnnn”, which refers to the gene numbering assigned to the TIGR4 strain by Tettelin et al. [6]. This numbering unambiguously identifies any particular gene in the TIGR4 strain, and the gene's sequence and chromosomal location from the TIGR4 genome can readily be used to identify the corresponding gene in any other strain of S. pneumoniae. For ease of reference, the corresponding gene in the R6 genome [4] is also indicated.

Knockout Bacteria

The invention provides a S. pneumoniae bacterium in which expression of one or more of the genes listed in Tables 1 & 2 has been knocked out.

Techniques for gene knockout are well known, and knockout mutants of S. pneumoniae have been reported previously [e.g. refs. 7-11 etc.].

The knockout is preferably achieved using isogenic deletion of the coding region, but any other suitable technique may be used e.g deletion or mutation of the promoter, deletion or mutation of the start codon, antisense inhibition, inhibitory RNA, etc. In the resulting bacterium, however, mRNA encoding the gene product of Tables 1 & 2 will be absent and/or its translation will be inhibited (e.g. to less than 1% of wild-type levels).

The bacterium may contain a marker gene in place of the knocked out gene e.g an antibiotic resistance marker.

Screening Methods

The invention provides a process for determining whether a test compound down-regulates expression of a target polypeptide, comprising the steps of: (a) contacting the test compound with a S. pneumoniae bacterium to form a mixture; (b) incubating the mixture to allow the compound and the bacterium to interact; and (c) determining whether expression of the target polypeptide is down-regulated. The compound may act by inhibiting transcription or translation.

The invention also provides a process for determining whether a test compound binds to a target polypeptide, comprising the steps of: (a) contacting the test compound with the target polypeptide to form a mixture; (b) incubating the mixture to allow the compound and the target polypeptide to interact; and (c) determining whether the compound and polypeptide interact.

Where a target polypeptide is an enzyme, the invention also provides a process for determining whether a test compound inhibits the enzymatic activity of a target polypeptide, comprising the steps of: (a) contacting the test compound with the target polypeptide and a substrate for the enzymatic reaction catalysed by the target polypeptide; (b) incubating the mixture to allow the compound, target polypeptide and substrate to interact; and (c) determining whether modification of the substrate by the enzymatic activity is inhibited by the test compound.

The target polypeptide is preferably a S. pneumoniae polypeptide, and more preferably it is a S. pneumoniae polypeptide encoded by of one of the genes listed in Table 1 or Table 2 (or a polypeptide as specified in the middle column of Table 1 or Table 2). The polypeptide may be from any suitable strain e.g encoded by the pol.A gene from the 23F strain. The availability of sequence information for each of the genes listed in Tables 1 and 2 means that the skilled person will readily be able to identify a gene of interest in any strain of interest, if that identification has not already been made. For example, the sequence of the nadE gene from strain R6 (SPR1276) helps the skilled person to find the nadE gene in any other strain.

As an alternative, the target polypeptide comprises (a) an amino acid sequence having sequence identity to the amino acid sequence encoded by of one of the genes listed in Tables 1 & 2 and/or (b) an amino acid sequence comprising a fragment of the amino acid sequence encoded by of one of the genes listed in Tables 1 & 2. The polypeptide preferably retains the activity listed in Tables 1 & 2.

The degree of sequence identity is preferably greater than 50% (e.g 60%, 70%, 80%, 90%, 95%, 99% or more). These proteins include homologs, orthologs, allelic variants and finctional mutants of the Table 1 polypeptides. Identity between proteins is preferably determined by the Smith-Waterman homology search algorithm as implemented in the MPSRCH program (Oxford Molecular), using an affine gap search with parameters gap open penalty=12 and gap extension penalty=1.

The fragment should comprise at least n consecutive amino acids from the sequences and, depending on the particular sequence, n is 7 or more (e.g. 8, 10, 12, 14, 16, 18, 20, 30, 40, 50, 60, 70, 80, 90, 100 or more). Preferably the fragment comprises one or more epitopes from the sequence. The fragment may be a Table 1 polypeptide without one or more of its N-terminal amino acids e.g. lacking the N-terminus methionine and/or the N-terminus signal peptide.

As a further alternative, the polypeptide may be the homolog of a Table 1 polypeptide from another Streptococcus (such as S. pyogenes or S. agalactiae) or from another Gram-positive bacterium.

Polypeptides for use in the process of the invention can be prepared by various means (e.g. recombinant expression, purification from S. pneumoniae, chemical synthesis, etc.) and in various forms (e.g. native, fusions, non-glycosylated, etc.). As reagents, they are preferably used in substantially pure form (Lie. substantially free from other streptococcal or host cell proteins). The polypeptide may be immobilised on a support, either covalently or non-covalently. Polypeptides can be coated directly onto supports, or can be attached indirectly e.g. by the use of non-neutralising antibodies which are themselves attached to the support.

The test compound may be of extracellular, intracellular, biologic or chemical origin. Typical test compounds include peptide, peptoids, lipids, nucleotides, nucleosides, small organic molecules, antibiotics, polyamines, polymers, or derivatives thereof. Small organic molecules have a molecular weight of between 50 and 2500 Da, and most preferably between about 300 and about 800 Da.

The test compound may be in a purified form, or may be part of a mixture of substances, such as extracts containing natural products, or the products of mixed combinatorial syntheses. Test compounds may be derived from large libraries of synthetic or natural compounds. For instance, synthetic compound libraries are commercially available, as are libraries of natural compounds in the form of bacterial, fungal, plant and animal extracts. If a mixture is found to have a useful activity then that activity can then be traced to specific component(s) either by knowing the components and testing them individually, or by purification or deconvolution. Additionally, test compounds may be synthetically produced using combinatorial chemistry either as individual compounds or as mixtures.

The screening method of the invention is preferably arranged in a high-throughput format. Conveniently, the method is performed in a microtitre plate.

If a test compound binds to a protein of the invention and this binding inhibits the life cycle of the S. pneumoniae bacterium, then the test compound can be used as an antibiotic or as a lead compound for the design of antibiotics.

Methods for detecting down-regulation of transcription are well known in the art, and the method of detection is not critical to the invention. Methods for detecting mRNA include, but are not limited to amplification assays such as quantitative RT-PCR, and/or hybridisation assays such as Northern analysis, dot blots, slot blots, in situ hybridisation, DNA assays, microarray, etc.

Methods for detecting down-regulation of translation are also well known in the art and, again, the method of detection is not critical to the invention. Methods of polypeptide detection include, but are not limited to, immunodetection methods such as Western blots, ELISA assays, polyacrylamide gel electrophoresis, mass spectroscopy, and enzymatic assays.

Methods for detecting a binding interaction are well known in the art and may involve techniques such as NMR, filter-binding assays, gel-retardation or gel-shift assays, displacement assays, western blots, radiolabeled competition assays, co-fractionation by chromatography, co-precipitation, cross linking, surface plasmon resonance, reverse two-hybrid, etc. A compound which is found to bind to a polypeptide can be tested for antibiotic activity by contacting the compound with S. pneumoniae (or another bacterium) and then monitoring for inhibition of growth.

Direct methods for detecting a binding interaction may involve a labelled test compound and/or polypeptide. The label may be a fluorophore, radioisotope, or other detectable label. Association of the label with the polypeptide indicates a binding interaction. Other direct methods for assessing interaction between the test compound and a target polypeptide may include using NMR to determine whether a polypeptide:compound complex is present.

Another method of assessing interaction between a polypeptide and a test compound may involve immobilising the polypeptide on a solid surface and assaying for the presence of free test compound. If there is no interaction between the test compound and the polypeptide then free test compound will be detected. The test compound may be labelled to facilitate detection. This type of assay may also be carried with the test compound being immobilised on the solid surface. Interaction between the immobilised polypeptide and the free test compound may also be monitored by a process such as surface plasmon resonance.

Methods for assessing inhibition of enzymatic activity are well known [e.g. ref. 12]. Enzyme substrates are widely available from commercial manufacturers, including those adapted for in vitro assays e.g. coloured substrates or products to give visible indications of enzymatic activity, etc.

In the processes of the invention, a reference standard is typically needed in order to detect whether a target polypeptide and a test compound interact, or to detect whether expression of a given target polypeptide has been inhibited, or to detect whether enzymatic activity is inhibited. One standard is a control experiment run in parallel to a process of the invention in the absence of the test compound. The results achieved in the control experiment and the process of the invention can then be compared in order to assess the effect of the test compound. As an alternative to determining the standard in parallel, it may have been determined before performing the process of the invention, or after the process has been performed. The standard may be an absolute standard derived from previous work.

Some embodiments of the invention comprise using competitive screening assays in which neutralising antibodies capable of binding a polypeptide of the invention specifically compete with a test compound for binding to the polypeptide. In this manner, the antibodies can be used to detect the presence of any peptide which shares one or more antigenic determinants with the S. pneumoniae polypeptide. Radiolabeled competitive binding studies are described in ref. 13.

In other embodiments, the S. pneumoniae polypeptides are employed as research tools for identification, characterisation and purification of interacting, regulatory proteins. Appropriate labels are incorporated into the polypeptides of the invention by various methods known in the art and the polypeptides are used to capture interacting molecules. For example, molecules are incubated with the labelled polypeptides, washed to remove unbound polypeptides, and the polypeptide complex is quantified. Data obtained using different concentrations of polypeptide are used to calculate values for the number, affinity, and association of polypeptide with the complex.

Compounds Identified by Screening Processes

Test compounds which down-regulate expression of and/or which bind to a target polypeptide and/or which inhibit an enzymatic activity are useful as antibiotics, antibiotic candidates, or lead compounds for antibiotic development. Once a test compound has been identified as a compound that binds to a target polypeptide, or which inhibits its expression in a bacterium, it may be desirable to perform further experiments to confirm the in vivo function of the compound in inhibiting bacterial growth. Any of the above processes may therefore comprise the further steps of contacting the test compound with a bacterium and assessing its effect on bacterial growth and/or survival. Methods for determining bacterial growth and survival are routinely available.

The invention provides a compound obtained or obtainable by any of the processes described above. Preferably, the compounds are organic compounds.

Once a compound has been identified using a process of the invention, it may be necessary to conduct further work on its pharmaceutical properties. For example, it may be necessary to alter the compound to improve its pharmacokinetic properties or bioavailability. The invention extends to any compounds identified by the methods of the invention which have been altered to improve their pharmacokinetic properties and/or bioavailability, and to composition comprising those compounds.

The invention further provides compounds obtained or obtainable using the processes of the invention, and compositions comprising those compounds, for use as a medicament e.g as an antibiotic. The invention also provides the use of compounds obtained or obtainable using the processes of the invention in the manufacture of an antibiotic, particularly an antibiotic for treating S. pneumoniae infection.

The invention also provides a method for producing an antibiotic composition, comprising the steps of: (a) identifying a compound as described above; (b) manufacturing the compound; (c) formulating the compound for administration to a patient; and (d) packaging the formulated compound to produce the antibiotic composition. Details of pharmaceutical formulation can be found in ref. 14.

Combinations of Polypeptides

The invention also provides a composition comprising mn or more polypeptides, wherein each of the m or more polypeptides is: (a) a S. pneumoniae polypeptide encoded by of one of the genes listed in Table 1 or Table 2 or as specified in the middle column of Table 1 or Table 2; (b) a polypeptide comprising (i) an amino acid sequence having sequence identity to the amino acid sequence encoded by of one of the genes listed in Tables 1 & 2 and/or (ii) an amino acid sequence comprising a fragment of the amino acid sequence encoded by of one of the genes listed in Tables 1 & 2; or (c) a homolog of a Table 1 polypeptide from another Streptococcus (such as S. pyogenes or S. agalactiae) or from another Gram-positive bacterium.

The invention also provides a hybrid polypeptide comprising the amino acid sequences ofp or more polypeptides as defined in (a), (b) or (c) above. Thus a plurality of the 101 polypeptides of the invention are expressed as a single polypeptide chain. Linker peptide sequences may be included between different members of the 101 polypeptides of the invention.

The values of m and of p are, independently, at least 2 (e.g. 2, 3, 4, 5, 6, 7, 8, 9, 10, 11, 12, 13, 14, 15, 16, 17, 18, 19, 20 or more).

The degree of sequence identity is preferably greater than 50% (e.g. 60%, 70%, 80%, 90%, 95%, 99% or more), as mentioned above. A fragment on (b)(ii) should comprise at least n consecutive amino acids from the sequences, as mentioned above.

Compositions and hybrid polypeptides of the invention are preferably immunogenic, and may be used for immunisation and vaccination purposes. Compositions may thus include an adjuvant, Suitable adjuvants include, but are not limited to: (A) aluminium salts, including hydroxides (e.g. oxyhydroxides), phosphates (e.g. hydroxyphoshpates, orthophosphates), sulphates, etc. [e.g. see chapters 8 & 9 of ref. 15]), or mixtures of different aluminium compounds, with the compounds taking any suitable form (e.g gel, crystalline, amorphous, etc.), and with adsorption being preferred; (B) MF59 (5% Squalene, 0.5% Tween 80, and 0.5% Span 85, formulated into submicron particles using a microfluidizer) [see Chapter 10 of 15; see also ref. 16]; (C) liposomes [see Chapters 13 and 14 of ref. 15]; (D) ISCOMs [see Chapter 23 of ref. 15], which may be devoid of additional detergent [17]; (E) SAF, containing 10% Squalane, 0.4% Tween 80, 5% pluronic-block polymer L121, and thr-MDP, either microfluidized into a submicron emulsion or vortexed to generate a larger particle size emulsion [see Chapter 12 of ref. 15]; (F) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™; (G) saponin adjuvants, such as QuilA or QS21 [see Chapter 22 of ref. 15], also known as Stimulon™ [18]; (n) chitosan [e.g. 19]; (I) complete Freund's adjuvant (CFA) and incomplete Freund's adjuvant (IFA); (J) cytokines, such as interleukins (e.g. IL-1, IL-2, IL-4, IL-5, IL-6, IL-7, IL-12, etc.), interferons (e.g. interferon-γ), macrophage colony stimulating factor, tumor necrosis factor, etc. [see Chapters 27 & 28 of ref. 15]; (K) monophosphoryl lipid A (MPL) or 3-O-deacylated MPL (3dMPL) [e.g. chapter 21 of ref. 15]; (L) combinations of 3dMPL with, for example, QS21 and/or oil-in-water emulsions [20]; (M) a polyoxyethylene ether or a polyoxyethylene ester [21]; (N) a polyoxyethylene sorbitan ester surfactant in combination with an octoxynol [22] or a polyoxyethylene alkyl ether or ester surfactant in combination with at least one additional non-ionic surfactant such as an octoxynol [23]; (N) a particle of metal salt [24]; (O) a saponin and an oil-in-water emulsion [25]; (P) a saponin (e.g. QS21)+3dMPL+IL-12 (optionally+a sterol) [26]; (Q) E. coli heat-labile enterotoxin (“LT”), or detoxified mutants thereof, such as the K63 or R72 mutants [e.g. Chapter 5 of ref. 27]; (R) cholera toxin (“CT”), or detoxified mutants thereof [e.g. Chapter 5 of ref. 27]; (S) double-stranded RNA; (T) microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) being preferred, optionally treated to have a negatively-charged surface (e.g. with SDS) or a positively-charged surface (e.g. with a cationic detergent, such as CTAB); (U) oligonucleotides comprising CpG motifs i.e. containing at least one CG dinucleotide, with 5-methylcytosine optionally being used in place of cytosine; (V) monophosphoryl lipid A mimics, such as aminoalkyl glucosaminide phosphate derivatives e.g. RC-529 [28]; (W) polyphosphazene (PCPP); (X) a bioadhesive [29] such as esterified hyaluronic acid microspheres [30] or a mucoadhesive selected from the group consisting of cross-linked derivatives of poly(acrylic acid), polyvinyl alcohol, polyvinyl pyrollidone, polysaccharides and carboxymethylcellulose; or (Y) other substances that act as immunostimulating agents to enhance the effectiveness of the composition [e.g. see Chapter 7 of ref. 15]. Aluminium salts are preferred adjuvants for parenteral immunisation. Mutant toxins are preferred mucosal adjuvants.

Muramyl peptides include N-acetyl-muramyl-L-threonyl-D-isoglutamine (thr-MDP), N-acetyl -normuramyl-L-alanyl-D-isoglutamine (nor-MDP), N-acetylmuramyl-L-alanyl-Disoglutaminyl-L-alanine-2-(1′-2′-dipalmitoyl-sn-glycero-3-hydroxyphosphoryloxy)ethylamine MTP-PE), etc.

The composition may also comprise other polypeptide or polysaccharide antigens e.g. from S. pneumoniae, from other bacteria, from other pathogens, etc. Inclusion of saccharide antigens (preferably conjugated) from Neisseria is convenient.

The composition may also include an antibiotic.

A summary of standard techniques and procedures which may be employed to perform the invention follows. This summary is not a limitation on the invention but, rather, gives examples that may be used, but are not required.

General

The practice of the present invention will employ, unless otherwise indicated, conventional techniques of molecular biology, microbiology, recombinant DNA, and immunology, which are within the skill of the art. Such techniques are explained fully in the literature eg. Sambrook Molecular Cloning; A Laboratory Manual, Second Edition (1989); DNA Cloning Volumes I and II (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

Definitions

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “comprising” means “including” as well as “consisting” e.g. a composition “comprising” X may consist exclusively of X or may include something additional e.g. X+Y.

The term “about” in relation to a numerical value x means, for example, x+10%.

The word “substantially” does not exclude “completely” e.g. a composition which is “substantially free” from Y may be completely free from Y. Where necessary, the word “substantially” may be omitted from the definition of the invention.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a streptococcus sequence is heterologous to a mouse host cell. A further examples would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (eg. 60%, 70%, 80%, 90%, 95%, 99% or more, calculated using the Smith-Waterman algorithm as described above). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions (eg. see U.S. Pat. No. 5,753,235).

Expression Systems

The streptococcus nucleotide sequences can be expressed in a variety of different expression systems; for example those used with mammalian cells, baculoviruses, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (eg. structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation [Sambrook et al. (1989) “Expression of Cloned Genes in Mammalian Cells.” In Molecular Cloning: A Laboratory Manual, 2nd ed.].

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter [Maniatis et al. (1987) Science 236:1237; Alberts et al. (1989) Molecular Biology of the Cell, 2nd ed.]. Enhancer elements derived from viruses may be particularly useful, because they usually have a broader host range. Examples include the SV40 early gene enhancer [Dijkema et al (1985) EMBO J. 4:761] and the enhancer/promoters derived from the long terminal repeat (LTR) of the Rous Sarcoma Virus [Gorman et al. (1982b) Proc. Natl. Acad. Sci. 79:6777] and from human cytomegalovirus [Boshart et al. (1985) Cell 41:521]. Additionally, some enhancers are regulatable and become active only in the presence of an inducer, such as a hormone or metal ion [Sassone-Corsi and Borelli (1986) Trends Genet. 2:215; Maniatis et al. (1987) Science 236:1237].

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation [Birnstiel et al. (1985) Cell 41:349; Proudfoot and Whitelaw (1988) “Termination and 3′ end processing of eukaryotic RNA. In Transcription and splicing (ed. B. D. Hames and D. M. Glover); Proudfoot (1989) Trends Biochem. Sci. 14:105]. These sequences direct the transcription of an mRNA which can be translated into the polypeptide encoded by the DNA. Examples of transcription terminater/polyadenylation signals include those derived from SV40 [Sambrook, et al (1989) “Expression of cloned genes in cultured mammalian cells.” In Molecular Cloning: A Labortory Manual].

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (eg. plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 [Gluzman (1981) Cell 23:175] or polyomavirus, replicate to extremely high copy number in the presence of the appropriate viral T antigen. Additional examples of mammalian replicons include those derived from bovine papillomavirus and Epstein-Barr virus. Additionally, the replicon may have two replicaton systems, thus allowing it to be maintained, for example, in mammalian cells for expression and in a prokaryotic host for cloning and amplification. Examples of such mammalian-bacteria shuttle vectors include pM2 [Kaufinan et al. (1989) Mol. Cell. Biol. 9:946] and pHEBO [Shimizu et al. (1986) Mol. Cell. Biol. 6:1074].

The transformation procedure used depends upon the host to be transformed. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (eg. Hep G2), and a number of other cell lines.

ii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego CA (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence, and transcription termination sequence, are usually assembled into an intermediate transplacement construct (transfer vector). This may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extra-chromosomal element (e.g. plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (eg. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Yirol. 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell postaanslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon, Maeda et al., (1985), Nature 315:592; human gastrin-releasing peptide, Lebacq-Verheyden et al., (1988), Molec. Cell. Biol. 8:3129; human EL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is cotransformed with the heterologous DNA of the transfer vector and the genomic DNA of wild type baculovirus—usually by cotransfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skiled in the art. Namely, the plaques are screened under the light microscope for the presence (indicative of wild-type virus) or absence (indicative of recombinant virus) of occlusion bodies. “Current Protocols in Microbiology” Vol. 2 (Ausubel et al. eds) at 16.8 (Supp. 10, 1990); Summers and Smith, supra; Miller et al. (1989).

Recombinant baculovirus expression vectors have been developed for infection into several insect cells. For example, recombinant baculoviruses have been developed for, inter alia: Aedes aegypti , Autographa californica, Bombyx mori, Drosophila melanogaster, Spodoptera frugiperda, and Trichoplusia ni (WO 89/046699; Carbonell et al., (1985) J. Virol 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, eg. Summers and Smith supra.

The modified insect cells may then: be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, eg. HPLC, affinity chromatography, ion exchange chromatography, etc.; electrophoresis; density gradient centrifugation; solvent extraction, etc. As appropriate, the product may be further purified, as required, so as to remove substantially any insect proteins which are also present in the medium, so as to provide a product which is at least substantially free of host debris, eg. proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the ark based upon what is known in the art.

iii. Plant Systems

There are many plant cell culture and whole plant genetic expression systems known in the art. Exemplary plant cellular genetic expression systems include those described in patents, such as: U.S. Pat. Nos. 5,693,506; 5,659,122; and 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMlin, Gibberellins: in: Advanced Plant Physiology,. Malcolm B. Wilkins, ed., 1984 Pitman Publishing Limited, London, pp. 21-52. References that describe other metabolically-regulated genes: Sheen, Plant Cell, 2:1027-1038(1990); Maas et al., EMBO J. 9:3447-3452 (1990); Benkel and Hickey, Proc. Natl. Acad. Sci. 84:1337-1339 (1987).

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11(2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed.

While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaila, Lotus, Medicago, Onobrychis, Trifoliumn, Trigonella, Vigna, Citrus, Linum, Geranium, Manihot, Daucus, Arabidopsis, Brassica, Raphanus, Sinapis, Atropa, Capsicunm, Datura, Hyoscyamus, Lycopersion, Nicotiana, Solanum, Petunia, Digitalis, Majorana, Cichorium, Heliantlhus, Lactuca, Bronmus, Asparagus, Antilrhinum, Hererocallis, Nemesia, Pelargonium, Panicum, Pennisetum, Ranunculus, Senecio, Salpiglossis, Cucumis, Browaalia, Glycine, Lolium, Zea, Triticuin, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacteral Systems

Bacterial expression techniques are known in the art. A bacterial promoter is any DNA sequence capable of binding bacterial RNA polymerase and initiating the downstream (3′) transcription of a coding sequence (eg. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al. (1984) Annu. Rev. Genet. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (trp) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The g-laotamase (bla) promoter system [Weissmann (1981) “The cloning of interferon and other mistakes.” In Interferon 3 (ed. I. Gresser)], bacteriophage lambda PL [Shimatake et al. (1981) Nature 292:128] and T5 [U.S. Pat. No. 4,689,406] promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid trp-lac promoter comprised of both trp promoter and lac operon sequences that is regulated by the lac repressor [Amann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EPO-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon [Shine et al. (1975) Nature 254:34]. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA.” In Biological Regulation and Development: Gene Expression (ed. R. F. Goldberger)]. To express eukaryotic genes and prokaryotic genes with weak ribosome-binding site [Sambrook et al. (1989) “Expression of cloned genes in Escherichia coli.” In Molecular Cloning. A Laboratory Manual].

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EPO-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleaye the bacteriophage protein from the foreign gene [Nagai et al. (1984) Nature 309:810]. Fusion proteins can also be made with sequences from the lacZ [Jia et al. (1987) Gene 60:197], trpE [Allen et al. (1987) J. Biotechnol. 5:93; Makoff et al. (1989) J. Gen. Microbiol. 135:11], and Chey [EP-A-0 324 647] genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (eg. ubiquitin specific processing-protease) to cleave the ubiquitin from the foreign protein. Through this method, native foreign protein can be isolated [Miller et al. (1989) Bio/Technology 7:698].

Alternatively, foreign proteins can also be secreted from the cell by creating chimeric DNA molecules that encode a fusion protein comprised of a signal peptide sequence fragment that provides for secretion of the foreign protein in bacteria [U.S. Pat. No. 4,336,336]. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb et al. (1984) EMBO J. 3:2437] and the E. coli alkaline phosphatase signal sequence (phoA) [Oka et al. (1985) Proc. Natl. Acad. Sci. 82:7212]. As an additional example, the signal sequence of the alpha-amylase gene from various Bacillus strains can be used to secrete heterologous proteins from B. subtilis [Palva et al. (1982) Proc. Natl. Acad. Sci. USA 79:5582; EP-A-0 244 042].

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stern loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (eg. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recoinbinations between homologous DNA in the vector and the bacterial chromosome. For example, integrating vectors constructed with DNA from various Bacillus strains integrate into the Bacillus chromosome (EP-A-0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Annu. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria For example, expression vectors have been developed for, inter alia, the following bacteria: Bacillus subtilis [Palva et al. (1982) Proc. Natl. Acad. Sci. USA 79:5582; EP-A-0 036 259 and EP-A-0 063 953; WO 84/04541], Escherichia coli [Shimatake et al. (1981) Nature 292:128; Amann etal. (1985) Gene 40:183; Studier etal. (1986) J. Mol. Biol 189:113; EP-A-0 036 776, EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. See eg. [Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus], [Miller et al. (1988) Proc. Natl. Acad. Sci. 85:856; Wang et al. (1990) J. Bacteriol. 172:949, Campylobacter], [Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids. In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus].

v. Yeast Expression

Yeast expression systems are also known to one of ordinary skill in the art. A yeast promoter is any DNA sequence capable of binding yeast RNA polymerase and initiating the downstream (3′) transcription of a coding sequence (eg. structural gene) into mRNA. A promoter will have a transcription initiation region which is usually placed proximal to the 5′ end of the coding sequence. This transcription initiation region usually includes an RNA polym erase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PHO5 gene, encoding acid phosphatase, also provides useful promoter sequences [Myanohara et al. (1983) Proc. Natl. Acad. Sci. USA 80:1].

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (EP-A-0 164 556). Furthermore, a yeast promoter can include naturally occurring promoters of non-yeast origin that have the ability to bind yeast RNA polymerase and initiate transcription.

Examples of such promoters include, inter alia, [Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109;].

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See eg. EP-A-0 196 056. Another example is a ubiquitin fusion protein. Such a fusion protein is made with the ubiquitin region that preferably retains a site for a processing enzyme (eg ubiquitin-specific processing protease) to cleave the ubiquitin from the foreign protein. Through this method, therefore, native foreign protein can be isolated (eg. WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the yeast invertase gene (EP-A-0 012 873; JPO. 62,096,086) and the A-factor gene (U.S. Pat. No. 4,588,684). Alternatively, leaders of non-yeast origin, such as an interferon leader, exist that also provide for secretion in yeast (EP-A-0 060 057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alphafactor. (eg. see WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (eg plasmids) capable of stable maintenance in a host, such as yeast or bacteria The replicon may have two replication systems, thus allowing it to be maintained, for example, in yeast for expression and in a prokaryotic host for cloning and amplification. Examples of such yeast-bacteria shuttle vectors include YEp24 [Botstein et al. (1979) Gene 8:17-24], pCl/1 [Brake et al. (1984) Proc. Natl. Acad. Sci USA 81:4642-4646], and YRp17 [Stinchcomb et al. (1982) J. Mol. Biol. 158:157]. In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See eg. Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome [Orr-Weaver et al. (1983) Methods in Enzymol. 101:228-245]. An integrating vector may be directed to a specific locus in yeast by selecting the appropriate homologous sequence for inclusion in the vector. See Orr-Weaver et aL, supra. One or more expression construct may integrate, possibly affecting levels of recombinant protein produced [Rine et al. (1983) Proc. Natl. Acad. Sc. USA 80:6750]. The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions [Butt et al. (1987) Microbiol, Rev. 51:351].

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts: Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis [Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol 25:141], Pichia pastoris [Cregg, et al. (1985) Mol. Cell. Biol 5:3376; U.S. Pat. Nos. 4,837,148 and 4,929,555], Saccharomyces cerevisiae [Hinnen et al. (1978) Proc. Natl. Acad. Sci. USA 75:1929; Ito et al. (1983) J. Bacteriol. 153:163], Schizosaccbaromyces pombe [Beach and Nurse (1981) Nature 300:706], and Yarrowia lipolytica [Davidow, et al. (1985) Curr. Genet. 10:380471 Gaillardin, et al. (1985) Curr. Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed. See eg. [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al. (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 and 4,929,555; Pichia]; [Hinnen et al. (1978) Proc. Natl. Acad. Sci. USA 75;1929; Ito et al. (1983) J. Bacteriol. 153:163 Saccharomyces]; [Beach and Nurse (1981) Nature 300:706; Schizosaccharomyces]; [Davidow et al. (1985) Curr. Genet. 10:39; Gaillardin et al. (1985) Curr. Genet. 10:49; Yarrowia].

Antibodies

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

Antibodies against the proteins of the invention are useful for affinity chromatography, immunoassays, and distinguishing/identifying streptococcus proteins.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera is obtained by bleeding the immunized animal into a glass or plastic container, incubating the blood at 25° C. for one hour, followed by incubating at 4° C. for 2-18 hours. The serum is recovered by centrifugation (eg. 1,000 g for 10 minutes). About 20-50 ml per bleed may be obtained from rabbits.

Monoclonal antibodies are prepared using the standard method of Kohler & Milstein [Nature (1975) 256:495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (eg. hypoxanthine, aminopterin, thymidine medium, “HAT”). The resulting hybridomas are plated by limiting dilution, and are assayed for production of antibodies which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the instant invention.

Pharmaceutical Compositions

Pharmaceutical compositions can comprise either polypeptides, antibodies, or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, nasal, and transdernal or transcutaneous applications (eg. see WO98/20734), needles, and gene guns or hyposprays.

The nature of any carriers or other ingredients included in compositions will depend on the specific route of administration and particular embodiment of the invention to be administered. Antibiotics, for example, exist in various formulations.

Dosage of low molecular weight compounds will depend on the disease state or condition to be treated and other clinical factors such as weight and condition of the human or animal and the route of administration of the compound. For treating human or animals, between approximately 0.5 mg/kg of body weight to 500 mg/kg of body weight of the compound can be administered. Therapy is typically administered at lower dosages and is continued until the desired therapeutic outcome is observed.

Dosage treatment may be a single dose schedule or a multiple dose schedule.

Polynucleotide and Polypeptide Pharmaceutical Compositions

In addition to the pharmaceutically acceptable carriers and salts described above, the following additional agents can be used with polynucleotide and/or polypeptide compositions.

A. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

B. Hormones, Vitamins, etc.

Other groups that can be included are, for example: hormones, steroids, androgens, estrogens, thyroid hormone, or vitamins, folic acid.

C. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

D. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Feigner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner supra). Other commercially available liposomes include transfectace (DDAB/DOPE) and DOTAP/DOPE (Boerhinger). Other cationic liposomes can be prepared from readily available materials using techniques well known in the art. See, eg. Szoka (1978) Proc. Natl. Acad. Sci. USA 75:4194-4198; WO90/11092 for a description of the synthesis of DOTAP (1,2-bis(oleoyloxy)-3-(trimethylamrnonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See eg. Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

E. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C & E, over time these lipoproteins lose A and acquire C & E. VLDL comprises A, B, C & E apoproteins, LDL comprises apoprotein B; and HDL comprises apoproteins A, C, & E.

The amino acid of these apoproteins are known and are described in, for example, Breslow (1985) Annu Rev. Biochem 54:699; Law (1986) Adv. Exp Med. Biol. 151:162; Chen (1986) J. Biol Chem 261:12918; Kane (1980) Proc Natl Acad. Sci USA 77:2465; and Utermann (1984) Hum Genet 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth Enzymol. (supra); Pitas (1980) J. Biochem. 255:5454-5460 and Mahey (1979) J Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443. Lipoproteins can also be purchased from commercial suppliers, such as Biomedical Techniologies, Inc., Stoughton, Mass., USA. Further description of lipoproteins can be found in WO98/06437.

F. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (X174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spernmine, spermidine, and purtrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene. Lipofectin™, and lipofectAMINE™ are monomers that form polycationic complexes when combined with polynucleotides/polypeptides.

### MODES FOR CARRYING OUT THE INVENTION

Isogenic deletion mutants of clinical isolate strain D39 of S. pneumoniae (serotype 2) were prepared using Overlap Extension [Amberg et al. (1995) Yeast 11: 1275-1280] for several S. pneumoniae genes to assess the effect of deletion on viability. Precise gene disruptions were achieved by gene splicing following a “double fusion” PCR strategy. Each process was accomplished with a total of five PCR reactions: three standard PCR amplifications and two fusion PCR reactions. The first step was performed by amplifying an upstream (fragment U, primers: F1+R2) and a downstream region (fragment D, primers: F5+R6) for each gene to disrupt, plus a selectable marker sequence (fragment K, primers: F3+R4) to replace the gene's reading frame in between. The aphA-3 gene (kanamycin resistance) was chosen as universal K fragment for all mutant constructs. It was amplified in order to contain 24 bp 5′ and 3′ tails showing complementary sequence to U-3′ and D-5′ ends, respectively. A first fusion PCR was performed to link D to K. Each KD amplified fragment was then gel purified and a second fusion PCR reaction was performed in order to fuse it to the corresponding U fragment. Final chimera products constitute for gene disruption cassettes (UKD). During the final fusion PCR in the presence of primers F1 and R6, they were amplified by AmpliTaq polymerase (Applera) able to add a single deoxyadenosine to the 3′ ends of both DNA strands. Each construct was ligated into a pGEM-T Easy vector (Promega) endowed of single 3′-T overhangs at the insertion site and then introduced by electroporation into E. coli DH10B bacteria (Invitrogen). Plasmid minipreps were retrieved from true recombinant colonies and the rightness of chimeric inserts was confirmed by PCR. Plamid DNAs were used to transform Sp using synthetic CSP-1 to induce natural competence [Havarstein et al. (1995) 92:11140-44]. Briefly, early log phase D39 cultures (OD₆₀₀=0.05-0.1) were diluted 1:10 with brain heart infusion broth (BHIB) supplemented with 100 ng/ml CSP-1, 10 mM glucose and 10% inactivated horse serum (Sigma) and incubated for 15 min at 37° C. and 5% CO₂ without aeration. Plasmid DNA (1 μg) was added and samples were incubated for 1 h before being spread on selective blood agar plates (tryptic soy agar, TSA-Difco, supplemented with 3% defibrinated sheep blood and 500 μg/ml of kanamycin). Growth was allowed for 1-2 days at 37° C. in an atmosphere of 5% CO₂. Five to ten KanR CFUs were screened for each sample either by PCR (primer F1+R6) or by direct sequencing of chromosomal DNA to choose the correct isogenic mutant colony.

Knockout of any of the 91 genes listed in Table 1 resulted in no growth, indicating that the genes are essential for pneumococcal viability. Knockout of any of the 10 genes listed in Table 2 gave bacteria which had poor growth characteristics when cultured in the absence of blood. In contrast, knockout of any of the genes listed in Table 3 had no effect on growth phenotype.

It will be understood that the invention has been described by way of example only and modifications may be made whilst remaining within the scope and spirit of the invention.

TABLE 1
91 genes for which knockout is lethal in TIGR4 strain

| TIGR4 gene | TIGR4 annotation | R6 gene |
|---|---|---|
| SP0005 | peptidyl-tRNA hydrolase (pth) | spr0005 |
| SP0032 | DNA polymerase I (polA) | spr0032 |
| SP0047 | phosphoribosylformylglycinamide cyclo-ligase (purM) | spr0048 |
| SP0056 | adenylosuccinate lyase (purB) | spr0056 |
| SP0092 | ABC transporter, substrate-binding protein | spr0083 |
| SP0102 | glycosyl transferase | spr0091 |
| SP0103 | capsular polysaccharide biosynthesis protein, putative | spr0092 |
| SP0253 | glycerol dehydrogenase (gldA) | spr0234 |
| SP0261 | undecaprenyl diphosphate synthase (uppS) | spr240 |
| SP0289 | dihydropteroate synthase | spr0266 |
| SP0290 | dihydrofolate synthetase (folC) | spr267 |
| SP0292 | bifunctional folate synthesis protein (sulD) | spr269 |
| SP0336 | penicillin-binding protein 2X (pbpX) | spr304 |
| SP0337 | phospho-N-acetylmuramoyl-pentapeptide-transferase (mraY) | spr305 |
| SP0381 | mevalonate kinase (mvaK1) | spr338 |
| SP0382 | diphosphomevalonate decarboxylase (mvaD) | spr339 |
| SP0383 | phosphomevalonate kinase (mvaK2) | spr340 |
| SP0397 | mannitol-1-phosphate 5-dehydrogenase (mtlD) | spr359 |
| SP0402 | signal peptidase I (spi) | spr364 |
| SP0418 | acyl carrier protein (acpP) | spr378 |
| SP0420 | malonyl CoA-acyl carrier protein transacylase (fabD) | spr380 |
| SP0423 | acetyl-CoA carboxylase, bitoin carboxyl carrier protein (accB) | spr0383 |
| SP0425 | acetyl-CoA carboxylase, biotin carboxylase (accC) | spr0385 |
| SP0477 | 6-phospho-beta-galactosidase (lacG-1) | sp424 |
| SP0516 | heat shock protein GrpE (grpE) | spr454 |
| SP0529 | BlpC ABC transporter (blpB) | spr0466/0467 |
| SP0605 | fructose-bisphosphate aldolase (fba) | spr530 |
| SP0655 | sodium/hydrogen exchanger family protein | spr0573 |
| SP0656 | hypothetical protein | spr0573 |
| SP0669 | thymidylate synthase (thyA) | spr585 |
| SP0680 | ribosomal small subunit pseudouridine synthase A (rsuA-2) | spr597 |
| SP0689 | UDP-N-acetylglucosamine-N-acetylmuramyl-(pentapeptide)pyrophosphoryl- | spr0604 |
| | undecaprenol N-acetylglucosamine transferase (murG) | |
| SP0708 | amino acid ABC transporter, amino acid-binding protein, | spr0621 |
| | authentic frameshift | |
| SP0756 | cell division ABC transporter, ATP-binding protein FtsE (ftsE) | spr0666 |
| SP0757 | cell division ABC transporter, permease protein FtsX (ftsX) | spr0667 |
| SP0762 | S-adenosylmethionine synthetase (metK) | spr671 |
| SP0806 | DNA gyrase subunit B (gyrB) | spr715 |
| sp0839 | pantothenate kinase (coaA) | spr741 |
| SP0865 | DNA polymerase III, gamma and tau subunits (dnaX) | spr769 |
| SP0876 | 1-phosphofructokinase, putative | spr779 |
| SP0935 | thymidylate kinase (tmk) | spr835 |
| SP0944 | uridylate kinase (pyrH) | spr845 |
| SP0945 | ribosome recycling factor (frr) | spr846 |
| SP0974 | preprotein translocase, SecG subunit, putative | spr877 |
| SP0988 | UDP-N-acetylglucosamine pyrophosphorylase (glmU) | spr891 |
| SP1067 | cell division protein FtsW, putative | spr0973 |
| SP1079 | GTP-binding protein, GTP1/Obg family | spr984 |
| SP1084 | methionine aminopeptidase, type I (map) | spr992 |
| SP1117 | DNA ligase, NAD-dependent (ligA) | spr1024 |
| SP1128 | enolase (eno) | spr1036 |
| SP1263 | DNA topoisomerase I (topA) | spr1141 |
| SP1267 | licC protein (licC) | spr1145 |
| SP1268 | licB protein (licB) | spr1146 |
| SP1269 | choline kinase (pck) | spr1147 |
| sp1271 | cytidine diphosphocholine pyrophosphorylase, putative | spr1149 |
| SP1272 | polysaccharide biosynthesis protein, putative | spr1150 |
| sp1273 | licD1 protein (licD1) | spr1151 |
| SP1329 | N-acetylneuraminate lyase | spr1186 |
| SP1360 | homoserine kinase (thrB) | spr1218 |
| SP1366 | glycosyl transferase, group 1 | spr1224 |
| sp1367 | licD3 protein (licD3) | spr1225 |
| SP1390 | UDP-N-acetylenolpyruvoylglucosamine reductase (murB) | spr1247 |
| SP1420 | NH(3)-dependent NAD(+) synthetase (nadE) | spr1276 |
| SP1456 | polypeptide deformylase (def-1) | spr1310 |
| SP1458 | thioredoxin reductase (trxB) | spr1312 |
| SP1492 | cell wall surface anchor family protein | spr1345 |
| SP1521 | UDP-N-acetylmuramate--alanine ligase (murC) | spr1373 |
| SP1529 | polysaccharide biosynthesis protein, putative | spr1383 |
| SP1530 | UDP-N-acetylmuramoylalanyl-D-glutamate--2,6-diaminopimelate ligase (murE) | spr1384 |
| SP1534 | inorganic pyrophosphatase, manganese-dependent (ppaC) | spr1389 |
| SP1559 | phosphoglucomutase/phosphomannomutase family protein | spr1417 |
| SP1571 | dihydrofolate reductase (folA) | spr1429 |
| SP1589 | Mur ligase family protein | spr1443 |
| SP1610 | Bcl-2 family protein | spr1463 |
| SP1655 | phosphoglycerate mutase (gpmA) | spr1499 |
| SP1667 | cell division protein FtsA (ftsA) | spr1511 |
| SP1670 | UDP-N-acetylmuramoylalanyl-D-glutamyl-2,6-diaminopimelate- | spr1514 |
| | D-alanyl-D-alanyl ligase (murF) | |
| SP1690 | ABC transporter, substrate-binding protein | spr1534 |
| SP1698 | alanine racemase (alr) | spr1540 |
| SP1699 | holo-(acyl-carrier protein) synthase (acpS) | spr1541 |
| SP1709 | phosphoglycerate dehydrogenase-related protein | spr1553 |
| sp1726 | 3-hydroxy-3-methylglutaryl-CoA reductase | spr1570 |
| SP1735 | methionyl-tRNA formyltransferase (fmt) | spr1580 |
| SP1814 | indole-3-glycerol phosphate synthase (trpC) | spr1634 |
| SP1881 | glutamate racemase (murl, glr) | spr1696 |
| SP1906 | chaperonin, 60 kDa (groEL) | spr1722 |
| SP1907 | chaperonin, 10 kDa (groES) | spr1723 |
| sp1968 | phosphopantetheine adenylyltransferase (coaD) | spr1783 |
| SP1975 | SpoIIIJ family protein | spr1790 |
| SP2012 | glyceraldehyde 3-phosphate dehydrogenase (gap) | spr1825 |
| SP2216 | secreted 45 kd protein (usp45) | spr2021 |

TABLE 2
10 genes for which knockout results in poor
growth characteristics in TIGR4 strain

| TIGR4 | | |
|---|---|---|
| gene | TIGR4 annotation | R6 gene |
| SP0417 | 3-oxoacyl-(acyl-carrier-protein) synthase III | spr377 |
| | (fabH) | |
| SP0419 | enoyl-(acyl-carrier-protein) reductase (fabK) | spr0379 |
| SP0424 | (3R)-hydroxymyristoyl-(acyl-carrier-protein) | spr384 |
| | dehydratase (fabZ) | |
| SP0969 | GTP-binding protein Era (era) | spr0871 |
| SP1161 | acetoin dehydrogenase complex, E3 component, | spr1048 |
| | dihydrolipoamide dehydrogenase, putative | |
| SP1649 | manganese ABC transporter, permease protein, | spr1493 |
| | putative, authentic frameshift (psaC) | |
| SP1650 | manganese ABC transporter, manganese-binding | spr1494 |
| | adhesion liprotein (psaA) | |
| SP2047 | conserved domain protein | spr1858 |
| SP2051 | competence protein CglC (cglC) | spr1862 |
| SP2146 | conserved hypothetical protein | spr1954 |

NB: where the annotation specifies an “. . . ase”, the polypeptide generally has enzymatic activity.

TABLE 3
Genes for which knockout does not affect in vitro growth
characteristics of TIGR4
TIGR4 gene

SP0004
SP0010
SP0013
SP0014/2006
SP0034
SP0037
SP0041
SP0042
SP0043
SP0044
SP0045
SP0046
SP0048
SP0053
SP0054
SP0057
SP0060
SP0075
SP0079
SP0082
SP0098
SP0104
SP0105 + 0106
SP0107
SP0109
SP0112
SP0117
SP0129
SP0135
SP0148
SP0149
SP0150
SP0155
SP0175
SP0176
SP0177
SP0178
SP0185
SP0187
SP0191
SP0198
SP0199
SP0202
SP0205
SP0231
SP0251
SP0263
SP0266
SP0268
SP0278
SP0281
SP0284
SP0314
SP0317
SP0318
SP0322
SP0347
SP0350
SP0360
SP0366
SP0368
SP0369
SP0377
SP0378
SP0386
SP0390
SP0391
SP0400
SP0403
SP0406
SP0410
SP0413
SP0421
SP0422
SP0435
SP0439
SP0447
SP0457
SP0459
SP0483
SP0494
SP0498
SP0502
SP0526
SP0545
SP0585
SP0589
SP0599
SP0601
SP0603
SP0607
SP0611
SP0614
SP0615
SP0616
sp0615-sp0616
SP0617
SP0620
SP0623
SP0625
SP0627
SP0629
SP0637
SP0641
SP0648
SP0659/1000
SP0660
SP0664
SP0667
SP0671
SP0672
SP0678
SP0690
SP0694
SP0717
SP0718
SP0724
SP0725
SP0726
SP0730
SP0745
SP0746
SP0749
SP0758
SP0764
SP0766
SP0771
SP0785
SP0797
SP0804
SP0820
SP0825
SP0829
SP0834
SP0845
SP0858
SP0859
SP0860
SP0872
SP0873
sp0881
SP0894
SP0899
SP0907
SP0916
SP0920
SP0928
SP0929
SP0930
SP0931
SP0932
SP0938
SP0965
SP0966
SP0968
SP0975
SP0977
SP0979
SP0981
SP0991
SP0998
SP1000/0659
SP1002
SP1003/1174
SP1008
SP1013
SP1014
sp1017
SP1018
SP1024
SP1026
SP1032
SP1033
SP1046
SP1068
SP1069
sp1075
SP1087
SP1100
SP1112
SP1118
SP1122
SP1124
SP1154
SP1156
SP1166
SP1167
SP1168
SP1174/1003
SP1175
SP1176
sp1190
SP1191
sp1192
SP1193
SP1200
SP1202
SP1204
SP1208
SP1218
SP1225
SP1232
SP1243
SP1244
SP1274
SP1283
SP1284
SP1287
SP1298
SP1308
SP1330/1685
SP1342
SP1343
SP1359
SP1361
SP1362
SP1369
SP1370
SP1371
sp1373
SP1374
sp1376
sp1377
SP1382
SP1386
SP1387
SP1388
SP1389
SP1392
SP1394
SP1400
SP1410
SP1412
SP1417
SP1427
SP1429
SP1445
SP1447
SP1449
SP1466
SP1469
SP1479
SP1480
SP1498
SP1500
SP1505
SP1527
SP1549
SP1551
SP1555
SP1557
SP1560
SP1573
SP1576
SP1580
SP1586
SP1591
SP1603
SP1608
SP1623
SP1634
SP1645
SP1647
SP1648
SP1651
SP1654
SP1672
SP1673
SP1676
SP1683
SP1685/1330
SP1687
SP1693
SP1695
SP1697
SP1700 + 1701
SP1707
SP1715
SP1721
SP1724
SP1778
SP1780
sp1795
SP1808
sp1811 + 1812
sp1813
sp1815
SP1816
SP1826
SP1829
SP1833
SP1839
SP1852
SP1865
SP1870
SP1872
SP1891
SP1894
SP1897
SP1898
SP1912
SP1923
SP1937
SP1940
SP1941
SP1942
SP1950
SP1953
SP1954/1955
SP1963
SP1964
SP1967
sp1970
SP1978
SP1981
SP1990
SP1992
SP1995
SP2006/0014
SP2010
SP2017
SP2029
sp2033
SP2041
SP2044
SP2050
SP2053
SP2056
SP2060
SP2063
SP2066
SP2086
SP2091
SP2092
SP2096
SP2098
SP2099
SP2101
SP2105
sp2107
SP2108
sp2126
SP2132
SP2136
SP2143
SP2144
SP2145
SP2148
SP2151
SP2153
sp2155
sp2158
SP2169
SP2171
SP2173
SP2175
SP2185
SP2187
SP2189
SP2190
SP2197
SP2201
SP2205
SP2218
SP2222
SP2224
SP2231
SP2235
SP2236
SP2237
SP2239

### REFERENCES

(The Contents of which are Hereby Incorporated in Full)

[1] GenBankNC_{—}004512.
[2] GenBank NC_{—}003440.
[3] GenBankNC_{—}003098
[4] Hoskins et al. (2001) J. Bacteriol. 183:5709-5717.
[5] GenBankNC_{—}003028.
[6] Tettelin et al. (2001) Science 293:498-506
[7] WO02/077021.
[8] Mollerach et al. (1998) J Exp Med 188:2047-56.
[9] Lee et al. (1998) Appl Environ Microbiol 64:4796-4802.
[10] U.S. Pat. No. 5,981,281.
[11] Kolkman et al. (1996) J Bacteriol 178:3736-3441.
[12] Eisenthal & Danson (eds) Enzyme Assays (Practical Approach Series) ISBN:0199638209 (2002).
[13] Lin et al. (1997) Antimicrobial Agents and Chemotherapy 41:2127-2131.
[14] Gennaro (2000) Remington: The Science and Practice of Pharmacy. 20th edition, ISBN: 0683306472.
[15] Vaccine design: the subunit and adjuvant approach (1995) eds. Powell & Newman. ISBN 0-306-
44867-X.
[16] WO90/14837.
[17] WO00/07621.
[18] WO00/62800.
[19] WO99/27960.
[20] European patent applications 0835318, 0735898 and 0761231.
[21] WO99/52549.
[22] WO01/21207.
[23] WO01/21152.
[24] WO00/23105.
[25] WO99/11241.
[26] WO98/57659.
[27] Del Giudice et al. (1998) Molecular Aspects of Medicine, vol. 19, number 1.
[28] Johnson et al. (1999) Bioorg Med Chem Lett 9:2273-2278.
[29] International patent application WO00/50078.
[30] Singh et al. (2001) J. Cont. Rele. 70:267-276.

1. A Streptococcus Pneumoniae bacterium in which expression of one or more of the following genes has been knocked out: SP0005, SP0032, SP0047, SP0056, SP0092, SP0102, SP0103, SP0253, SP0261, SP0289, SP0290, SP0292, SP0336, SP0337,SP0381, SP0382, SP0383, SP0397, SP0402, SP0417, SP0418, SP0419, SP0420,SP0423, SP0424, SP0425, SP0477, SP0516, SP0529, SP0605, SP0655, SP0656, SP0669, SP0680, SP0689, SP0708, SP0756, SP0757, SP0762, SP0806, SP0839, SP0865, SP0876, SP0935, SP0944, SP0945, SP0969, SP0974, SP0988, SP1067, SP1079, SP1084, SP1117, SP1128, SP1161, SP1263, SP1267, SP1268, SP1269, SP1271, SP1272, SP1273, SP1329, SP1360, SP1366, SP1367, SP1390, SP1420, SP1456, SP1458, SP1492, SP1521, SP1529, SP1530, SP1534, SP1559, SP1571, SP1589, SP1610, SP1649, SP1650, SP1655, SP1667, SP1670, SP1690, SP1698, SP1699, SP1709, SP1726, SP1735, SP1814, SP1881, SP1906, SP1907, SP1968, SP1975, SP2012, SP2047, SP2051, SP2146, and/or SP2216, wherein the SPnnnn nomenclature refers to the gene numbering assigned to the S. pneumoniae TIGR4 strain in Tettelin et al. (2001) Science 293:498-506.

2. The bacterium of claim 1, wherein expression is knocked out by isogenic deletion of the coding region of said gene (s).

3. The bacterium of claim 1, wherein the bacterium contains a marker gene in place of the knocked out gene.

4. A process for determining whether a test compound down-regulates expression of a target polypeptide, comprising the steps of: (a) contacting the test compound with a S. peumoniae bacterium of any one of claims 1 to 3, to form a mixture; (b) incubating the mixture to allow the compound and the bacterium to interact; and (c) determining whether expression of the target polypeptide is down-regulated, wherein the target polypeptide is selected from the group consisting of SP0005, SP0032, SP0047, SP0056, SP0092,SP0102,SP0103, SP0253, SP0261, SP0289, SP0290, SP0292, SP0336, SP0337, SP0381, SP0382, SP0383, SP0397, SP0402, SP0417, SP0418, SP0419, SP0420, SP0423, SP0424, SP0425, SP0477, SP0516, SP0529, SP0605, SP0655, SP0656, SP0669, SP0680, SP0689, SP0708, SP0756, SP0757, SP0762, SP0806, SP0839, SP0865, SP0876, SP0935, SP0944, SP0945, SP0969, SP0974, SP0988, SP1067, SP1079, SP1084, SP1117, SP1128, SP1161, SP1263, SP1267, SP1268, SP1269, SP1271, SP1272, SP1273, SP1329, SP1360, SP1366, SP1367, SP1390, SP1420, SP1456, SP1458, SP1492, SP1521, SP1529, SP1530, SP1534, SP1559, SP1571, SP1589, SP1610, SP1649, SP1650, SP1655, SP1667, SP1670, SP1690, SP1698, SP1699, SP1709, SP1726, SP1735,SP1814,SP1881, SP1906, SP1907, SP1968,SP1975, SP2012, SP2047, SP2051, SP2146,-and/or SP2216, wherein the SPnnnn nomenclature refers to the gene numbering assigned to the S. pneumoniae TIGR4 strain in Tettelin et al. (2001) Science 293:498-506.

5. A process for determining whether a test compound binds to a target polypeptide, comprising the steps of: (a) contacting the test compound with the target polypeptide to form a mixture; (b) incubating the mixture to allow the compound and the target polypeptide to interact; and (c) determining whether the compound and polypeptide interact, wherein the target polypeptide is selected from the group consisting of SP0005, SP0032, SP0047, SP0056, SP0092, SP0102, SP0103, SP0253, SP0261, SP0289, SP0290, SP0292, SP0336, SP0337, SP0381, SP0382, SP0383, SP0397, SP0402, SP0417, SP0418, SP0419, SP0420, SP0423, SP0424, SP0425, SP0477, SP0516, SP0529, SP0605, SP0655, SP0656, SP0669, SP0680, SP0689, SP0708, SP0756, SP0757, SP0762, SP0806, SP0839, SP0865, SP0876, SP0935, SP0944, SP0945, SP0969, SP0974, SP0988, SP1067, SP1079, SP1084, SP1117, SP1128, SP1161, SP1263, SP1267, SP1268, SP1269, SP1271, SP1272, SP1273, SP1329, SP1360, SP1366, SP1367, SP1390, SP1420, SP1456, SP1458, SP1492, SP1521, SP1529, SP1530, SP1534, SP1559, SP1571, SP1589, SP1610, SP1649, SP1650, SP1655, SP1667, SP1670, SP1690, SP1698, SP1699, SP1709, SP1726, SP1735, SP1814, SP1881, SP1906, SP1907, SP1968, SP1975, SP2012, SP2047, SP2051, SP2146, and/or SP2216, wherein the SPnnnn nomenclature refers to the gene numbering assigned to the S. pneumoniae TIGR4 strain in Tettelin et al. (2001) Science 293:498-506.

6. The process of claim 5, wherein the test compound comprises a peptoid, a lipid, a nucleotide, a nucleoside, a small organic molecule with a molecular weight between 50 and 2500 Da, an antibiotics, a polyamine, a polymer, or a peptide.

7. A compound obtainable by the process of any one of claims 5 or 6.

8. The process of claim 4, wherein the test compound comprises a peptoid, a lipid, a nucleotide, a nucleoside, a small organic molecule with a molecular weight between 50 and 2500 Da, an antibiotics, a polyamine, a polymer, or a peptide.

9. A compound obtainable by the process of claim 4.

10. A compound obtainable by the process of claim 8.
